(19) United States
(12) Patent Application Publication
(10) Pub. No.: US 2018/0092349 A1
(43) Pub. Date: Apr. 5, 2018

(54) HANDLING OF BIOLOGICAL SAMPLES

(71) Applicant: Genea IP Holdings Pty Limited, Sydney, New South Wales (AU)

(72) Inventors: Eduardo VOM, Box Hill (AU); Thomas BECKITT, Darlinghurst (AU); Alex KISELYOV, San Diego, CA (US); Teija Tuulikki PEURA, Erskineville (AU); Kim John GILIAM, Wakerley (AU)

(73) Assignee: Genea IP Holdings Pty Limited, Sydney, New South Wales (AU)

(21) Appl. No.: 15/562,481

(22) PCT Filed: Apr. 4, 2016
(86) PCT No.: PCT/AU2016/000113

§ 371 Date: Sep. 28, 2017

(30) Foreign Application Priority Data
Apr. 2, 2015 (AU) 2015901208

(51) Int. Cl. A01N 1/02; C12N 5/00; A61K 35/12; C12N 5/073; C12N 5/071

(52) U.S. Cl. CPC A01N 1/0226; C12N 5/0012; A61K 2035/128; C12N 5/0604; C12N 5/0608; A61K 35/12

(57) ABSTRACT

The present invention relates to the handling of biological samples, for example, the holding, manipulating and culturing of biological samples. In one form the invention provides an overlay encapsulant for an in vitro cell culture comprising a synthetic compound and in another aspect the invention provides methods of temporarily encapsulating an in vitro cell culture comprising a synthetic compound. The invention has use in relation to the culturing and more particularly the encapsulation of biological samples, such as for example zygotes, embryos, oocytes, stem cells, sperm located in a culturing space, relevant pluripotent derivative(s) and/or differentiated progeny, intact or dispersed tissue and/or intact organism(s).

### FIELD OF INVENTION

The present invention relates to the handling of biological samples, for example, the holding, manipulating and culturing of biological samples. It will be convenient to hereinafter describe the invention in relation to the culturing and more particularly the encapsulation of biological samples, such as for example zygotes, embryos, oocytes, stem cells, sperm located in a culturing space, relevant pluripotent derivative(s) and/or differentiated progeny, intact or dispersed tissue and/or intact organism(s). However, it should be appreciated that the present invention is not limited to that use, only.

### BACKGROUND ART

Throughout this specification the use of the word “inventor” in singular form may be taken as reference to one (singular) inventor or more than one (plural) inventor of the present invention.

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

Assisted Reproductive Technology (ART) is becoming increasingly important in developed countries as a means of assisted reproduction. Since the birth of the world's first “test tube baby” in the late 1970's, more than 5 million babies have been born worldwide via the use of modern ART procedures. Currently it is estimated that the annual number of In Vitro Fertilization (IVF) cycles in the world is greater than 1.5 million and growing, especially in developing countries.

IVF involves hormonal stimulation of a woman's ovaries in order to incite multiple eggs to mature. Carefully timed, just before ovulation, the mature eggs are retrieved from ovarian follicles by transvaginal ultrasound-guided needle aspiration. The number of retrieved eggs can vary from about 0 to about 40, although about 10 to about 20 eggs is more typical. The eggs are subsequently stored in a culture medium based on human fallopian tubal fluid and incubated at 37° C. before fertilisation either by co-incubation with sperm (IVF) or intracytoplasmic sperm injection (ICSI). In IVF, usually about 100,000 to about 200,000 sperm are added to the oocytes in a small volume of fertilisation media, or in ICSI, a single sperm is directly injected to the egg using a fine micropipette. Fertilization is confirmed about 12 to 20 hours later by the presence of a paternal (from sperm) and maternal (from egg) pronucleus. Fertilisation rates can vary between 0 and 100%, but about 60% to about 70% fertilisation rate is considered normal.

Fertilised embryos are then cultured in laboratory for about 2 to 6 days during which time they develop from 1-cell to greater than about 100 cells. The developed embryos are commonly transferred to the patient's uterus either at cleavage stage (usually about 4-8 cells at Day 2-3) or at blastocyst stage (>100 cells at Day 5) for implantation and gestation. Alternatively, embryos can be cryopreserved at either stage for later embryo transfer.

Handling of gametes and embryo outside the body requires an optimal microenvironment that supports cellular processes required for embryo survival and development. This is achieved through a combination of culture medium and optimal incubation conditions. Maintenance of correct temperature and internal pH (pHi) of embryos is especially critical, and achieved by keeping gametes and embryos in temperature (+37° C.) and gas controlled (about 5-6% CO₂ and about 5-20% O₂) incubators in appropriately buffered (for example bicarbonate buffered) culture media.

However, there is a necessity to remove embryos from these conditions for various IVF-related steps such as ICSI or evaluation which exposes embryos to ambient conditions. To protect embryos during removal from the controlled incubation environment, naturally occurring and extracted oils referred to as mineral oil is commonly used to form a layer on top of culture media, referred to as an overlay, during in vitro fertilization (IVF) procedures¹. This is known as the microdrop method and it facilitates embryo assessment, allows for culturing embryos in a small volume of media, protects gametes or embryos from environment during handling such as for example intracytoplasmic sperm injection (ICSI), assisted hatching and alike, provides stabilization of pH and temperature, alleviates osmotic fluctuations and overall, is linked with improved embryogenesis². For example, mineral oil has been reported to alter embryo growth by sequestering xenobiotics affecting embryos³. It has been noted that culture of multiple embryos in small volumes of media overlaid with mineral oil allows for elevated concentration of autocrine growth factors secreted by the embryo, yielding the enhanced rates of development. It is generally recognized that an overlay of mineral oil:
1. Provides for a physical barrier separating droplets of medium from the atmosphere and airborne pathogens;
2. Delays gas diffusion thus keeping pH, temperature, osmolality and oxygen concentration of the media at steady levels protecting the embryos from significant fluctuations in their microenvironment;
3. Prevents evaporation allowing for the use of nonhumidified incubators; prevents free diffusion of metabolic by-products including ammonia;
4. Removes lipid-soluble xenobiotics ¹ Brinster, R. L. A method for in vitro cultivation of mouse ova from two-cell blastocyst,” Exp. Cell. Res. 1963, 32, 205-208; Johnson, C. et. al. “The use of oil overlay for in vitro fertilization and culture,” Assisted Repr. Rev. 1994, 4, 198-201² Swain, J. E. et. al. “Microdrop preparation factors influence culture-media osmolality, which can impair mouse embryo preimplantation development,” Repr. BioMed. Online 2012, 24, 142-147; Mathur, J. “Enhanced somatic embryogenesis in Selinum candolii DC under a mineral oil overlay,” Plant Cell, Tissue and Organ Culture 1991, 27, 23-26³ Miller, K. F., et al. “Covering embryo cultures with mineral oil alters embryo growth by acting as a sink for an embryotoxic substance,” J. Assist. Repr. Genet. 1994, 11 (7), 342-345

There are other applications for mineral oil overlay in the area of cell based assays, for example stem cell assays, in vitro, cell-based, tissue culture and in vivo assays involving intact organisms.

The term, ‘mineral oil’ will hereinafter be taken as reference to liquid by-products of the refining of naturally occurring crude oil.

Despite the advantages set out above, there are several noted issues associated with the use of mineral oil for culturing embryos. In the extreme, the use of mineral oil overlay may result in a product recall when oil intended for IVF has proven to be unsuitable for the purpose⁴. Some of the issues include: i) undefined composition of mineral oil often presented as a complex mixture of chemicals, including impurities; ii) toxicity (including but not limited to; toxicity caused by poor purification and/or inadequate quality control and; toxicity acquired during transport and/or storage) associated with both endogenous components, for example, polyaromatic hydrocarbons, (poly)unsaturated organics, heteroaromatic substances and, products of exposure to sunlight, air/oxygen⁵. Notably, human serum albumin (HSA) and/or related additives have been noted to further increase the toxic effect of peroxidized oil, presumably via stabilizing and propagating formation of the reactive oxygen species⁶. Silicon oil has been introduced as an alternative to both mineral and specifically paraffin oils, however it was reported to be somewhat toxic to embryos presumably due to the Zn impurities.⁷ Several groups have reported a superior performance of paraffin oil, as opposed to other mineral oils, for embryonic development⁸. ⁴ Cook Medical Sydney IVF Culture Oil product recall 2012, see http://www.accessdata.fda.gov/scripts/cdrh/cfdocs/cfRes/res.cfm?ID=107466⁵ peroxidation, Otsuki, J. et. al. “Peroxidation of mineral oil used in droplet culture is detrimental to fertilization and embryo development,” Fertility and Sterility 2007, 88 (3), 741-743); Otsuki, J. et. al. “Damage of embryo development caused by peroxidized mineral oil and its association with albumin in culture,” Fertility and Sterility 2008, 91 (5), 1745-1749; Provo, M. B. and Herr, C. “Washed paraffin oil becomes toxic to mouse embryos upon exposure to sunlight,” Theriogenology 1998, 49 (1), 214; Eertmans, F. “Validation of potentiometric peroxide value (POV) assay for analysis of mineral oil with low oxidative content,” J. Chem. Pharm. Res. 2013, 5(11), 395-402⁶ Otsuki, et al “Peroxidized oil and albumin reactions in culture,” Fertility & Sterility 2009⁷ Erbach et al “Zinc is a possible toxic contaminant of silicon oil in microdrop cultures of preimplantation mouse embryos,” Human Reprod. 1995, 10, 3248-3254⁸ Zhu, B. et al “Optimization of in vitro culture conditions in B6CBF1 mouse embryos,” Reprod. Nutr. Dev. 2004, 44, 219-231

Multiple precautionary measures have been described in the literature in order to reduce or eliminate reactive and/or toxic impurities. For example, SAGE™ Oil for Tissue Culture has been reported to result from an extensive and therefore controlled refinement process from crude oil. The resulting product is screened for unsaturated carbon bonds susceptible to peroxidation, metals, sulphur derivatives and stabilizers that could be toxic to embryos. In a similar claim, application of a Vitrolife™ product OVOIL™⁹ based on the sterile filtered paraffin oil containing predominantly saturated paraffins resulted in significantly higher development rate to morula and blastocyst than mineral oil¹⁰. The rectified EmbryoMax™ filtered light mineral oil is available from EMD Millipore¹¹, GM501 mineral oil has been introduced by Gynemed¹². There is also LifeGuard™ Oil by LifeGlobal Group¹³. A ‘head-to-head’ comparison of several commercially available mineral oils on embryonic development has also been described¹⁴. As a general recommendation, numerous authors suggest to use refined paraffin oil(s) and require the actual manufacturer to test their culture oil thoroughly. Despite these recommendations, utilization of commercially available mineral including paraffin oils poses considerable toxic/teratogenic risks to embryos associated with:
i) general lack of standardized, well-regimented refinement and further purification protocol for mineral and/or paraffin oils whilst suitable for biological and/or medical use, they may lead to the initial presence of toxic chemical groups or the ability of the oil to acquire toxicity during transport and/or storage.
ii) analytical techniques (NMR, GC MS, HPLC) that do not allow for the reliable detection of trace amounts of xenobiotics including but not limited to polyaromatic hydrocarbons, (poly)unsaturated aliphatic and aromatic compounds, heterocyclic molecules, nonvolatile aromatic amines (for example, anilines) and phenols, sulphides, their oligomers and low molecular weight reactive polymers and other cytotoxic species;
iii) complex chemical composition of the paraffin/mineral oils may potentially result in varying biophysical, chemical properties of the oil, embryo viability and development outcome.¹⁵ ⁹ See http://www.vitrolife.com/en/Fertility/Products/OVOIL/washed¹⁰ Tae, J. C. et. al. J. Assist. Reprod. & Gen. 2005¹¹ See http://www.emdmillipore.com/US/en/product/EmbryoMax%C2%AE-Filtered-Light-Mineral-Oil,MM_NF-ES-005-C?isCountryEMD=yes&¹² See http://www.gynemed.de/GM501-Mineral-Oil.102+M52087573ab0.0.html¹³ See http://www.lifeglobal.com/asp/Products/ProductDetail.asp?ID=LGUA¹⁴ Linck, D. SIRT, Australia 2008¹⁵ Gary D. Smith, et. al. (eds.) Embryo Culture: Methods and Protocols, Methods in Molecular Biology, vol. 912 Springer Science+Business Media, LLC 2012

Due to the deficiencies noted above, there is an ongoing need for homogeneous, stable, chemically and biologically inert and readily available materials, preferably oils, exhibiting physical properties such as for example, surface tension, viscosity, ease of handling/feasibility, partition coefficient/miscibility, gas/liquid diffusion potential, etc, that are suitable for both laboratory and cGMP manipulation of biological samples such as embryos.

### SUMMARY OF INVENTION

It is an object of the embodiments described herein to overcome or alleviate at least one of the above noted drawbacks of the prior art or to at least provide a useful alternative to prior art.

In a first aspect of embodiments described herein a solution is provided by the inventor for replacement of a mineral oil encapsulant for biological samples, be that in overlay microdrop form or otherwise that is commonly comprised of numerous poorly characterized compounds with one or a combination of the following:
A well-defined chemical compound or mixture of compounds as described by conventional analytical techniques such as for example, NMR, HPLC, LCMS and others within the limit of detection and as exemplified by a i) regimented polymer(s) with well-defined chemical and/or biophysical properties, ii) small molecule(s), iii) inert gas(es) heavier than air. An inert gas like Ar is an example of an inert media that encapsulates biological sample.
An inert chemical compound or mixture of compounds that is/are not miscible, non-toxic and features necessary encapsulant properties;
A transparent encapsulant comprising a chemical compound or mixture of compounds that allows for monitoring of the screening media via conventional detection techniques as exemplified by any UV/UV-vis/IR light absorption/emission techniques and/or biophysical methods. In this sense screening media may be applicable to embryo culture, enzymatic assay, cell-/tissue-/intact-organism based detection techniques.
A compound or mixture of compounds adapted to encapsulate or overlay a biological sample and which can be used for monitoring deviations in the properties and composition of media contained underneath it. This includes, but is not limited to; pH, ammonia concentration, osmolarity and presence of reactive oxygen species or volatile organic compounds.
A compound or mixture of compounds that can be used to remove toxic substances from the media onto which it is overlaid.
A chemical compound or mixture of compounds adapted to be used as a supplement source containing vitamins, hormones, growth factors, nutrients, protectants, RedOx traps, amino acids and their derivatives, peptoids, peptides, proteins, antibodies and relevant derivatives, fragments and full length oligonucleotides and their synthetic derivatives.
A chemical compound or mixture of compounds adapted to be used for other screening/biological manipulations involving element-sensitive proteins, cells/cell cultures, multi-origin tissues/tissue cultures, intact organisms.

With the above in mind the present invention in one aspect of embodiments provides an overlay encapsulant for an in vitro cell culture comprising a synthetic compound.

In using the overlay encapsulant, the cell culture may comprise one or more cells in a culture media. Preferably, the one or more cells comprises at least one or a combination of:

ovum;

zygote;

embryo;

animal/human-derived embryonic stem cell(s);

relevant pluripotent derivative(s) and/or differentiated progeny;

intact or dispersed tissue and/or intact organism.

The synthetic compound is preferably a synthetic small molecule composition exhibiting unequivocal chemical composition as identified via conventional analytical techniques within limits of detection and comprising one or a combination of.

synthetic monomer(s);

oligomers or polymers;

chemical derivatives and/or copolymers of polyalphaolefins,

each exhibiting specific chemical, biophysical and spectroscopic properties.

Alternatively, the synthetic compound comprises at least one hydrocarbon, a modified hydrocarbon. The modified hydrocarbon may comprise a fluorinated hydrocarbon.

In a further aspect of embodiments, the synthetic compound comprises one or a combination of long-chained, short-chained and cyclic hydrocarbons. In this respect, the synthetic compound may comprises a combination of long-chained, short-chained and cyclic hydrocarbons in the mixture of 45% long-chained, 38% short-chained and 17% cyclic, respectively.

In another aspect of embodiments of the present invention there is provided a method for temporary encapsulation of an in vitro cell culture comprising the step of overlaying the cell culture with a synthetic compound. Preferably, the synthetic compound may be a synthetic oil.

In yet another aspect of embodiments of the present invention there is provided a method for temporary encapsulation of at least one of protein(s), DNA, RNA sequence(s), relevant construct(s) and/or derivative(s), chemically-modified or derived analogues thereof for in vitro, ex vivo and/or in vivo manipulation thereof, the method comprising the step of:

overlaying a manipulation and/or screening media utilised in the in vitro, ex vivo and/or in vivo manipulation with a synthetic compound. Preferably, the synthetic compound is a synthetic oil.

In yet another aspect of embodiments of the present invention there is provided an overlay encapsulant for an in vitro cell culture comprising a synthetic compound being a well-defined chemical compound as described by conventional analytical techniques comprising one of NMR, H PLC, LCMS within the limit of detection wherein the compound is exemplified by one of:

i) regimented polymer with well-defined chemical and/or biophysical properties,

ii) small molecule,

iii) inert gas heavier than air.

In still another aspect of embodiments, the present invention provides an overlay encapsulant for an in vitro cell culture comprising a synthetic compound and adapted to monitor deviations in the properties and composition of media encapsulated thereby. The monitored properties and composition of encapsulated media may comprise one or a combination of:

pH,

ammonia concentration,

osmolarity,

presence of reactive oxygen species, and

presence or volatile organic compounds.

In yet another aspect of embodiments, the present invention provides an overlay encapsulant for an in vitro cell culture comprising a synthetic compound in which the overlay encapsulant is adapted to be used as a supplement source comprising one or a combination of:

vitamins,

hormones,

growth factors,

nutrients,

protectants,

RedOx traps,

amino acids and their derivatives,

peptoids,

peptides,

proteins,

antibodies and relevant derivatives, fragments and full length oligonucleotides and their synthetic derivatives.

In yet another aspect of embodiments, the present invention provides an overlay encapsulant for an in vitro cell culture comprising a synthetic compound in which the overlay encapsulant is adapted to be used for screening or biological manipulations involving one or more of:

element-sensitive proteins,

cells or cell cultures,

multi-origin tissues or tissue cultures, and

intact organisms.

Within embodiments of the invention the overlay encapsulant comprises a synthetic compound being synthetic oil which is a fully-synthetic oil comprising a synthetic small molecule (monomer, standalone compound), oligomer or a polymer exhibiting unequivocal chemical composition as identified via conventional analytical techniques within limits of detection and comprising one or a combination of.

synthetic monomer(s);

oligomers/polymers;

chemical derivatives and/or copolymers of polyalphaolefins,

each exhibiting specific chemical, biophysical and spectroscopic properties.

Preferred embodiments provide an overlay encapsulant and its uses for an in vitro cell culture comprising one or a combination of the following:

a well-defined chemical composition media as described by conventional analytical techniques such as for example, NMR, HPLC, LCMS and others within the limit of detection and as exemplified by a i) regimented polymer with well defined chemical and/or biophysical properties, ii) small molecule, iii) inert gas heavier than air. Preferably in the form of an inert gas Ar is excluded.

an inert chemical composition media that is not miscible, non-toxic and features necessary encapsulant properties;

a transparent encapsulant comprising a chemical composition media that allows for monitoring of the screening media via conventional detection techniques as exemplified by any UV/UV-vis/IR light absorption/emission techniques and/or biophysical methods. In this sense screening media may be applicable to embryo culture, enzymatic assay, cell-/tissue-/intact-organism based detection techniques.

a chemical composition media adapted to be used as a feeder layer containing vitamins, hormones, growth factors, nutrients, protectants, RedOx traps, amino acids and their derivatives, peptoids, peptides, proteins, fragments and full length oligonucleotides and their synthetic derivatives.

a chemical composition media adapted to be used for other screening/biological manipulations involving element-sensitive proteins, cells/cell cultures, multi-origin tissues/tissue cultures, intact organisms.

Other aspects and preferred forms are disclosed in the specification and/or defined in the appended claims, forming a part of the description of the invention.

In essence, embodiments of the present invention stem from the realization that reliable control in the handling of biological samples can be facilitated with the use of a fully synthetic, completely characterized substance as exemplified by synthetic oil(s), or synthetic compounds comprising polymer, small molecule and/or heavier than air inert gas and exhibiting i) well defined chemical and physical criteria, ii) purity and safety, iii) feasibility and ease of handling, iv) compatibility with embryology- and/or general biological testing requirements. Such characterised substances provide a superior alternative to the commonly used ‘mineral oil’.

The described invention of embodiments described and envisaged herein is anticipated to be generally applicable to any animal/human developmental work dealing with cellular, tissue-based or embryonic development/proliferation. Representative examples of the potential markets that may benefit from the invention include any/all animal, human IVF establishments, hospitals and clinics, pharmaceutical and biotechnology companies dealing with both early research and development, preclinical and clinical aspects of work with embryos or related cultures, Academia including specialized research institutes, universities and consortia.

Key competitive advantages of this approach include:
1. Well-defined consistent chemical composition of a synthetic compound allowing for easy adaptation of protocols to clinical/cGMP environment;
2. Reproducibility, consistency, feasibility of the proposed application;
3. Chemical inertness, ie absence of ‘active reactives’, providing resistance to sunlight, temperature, air/oxygen, specialized media components;
4. Optimized (bio)physical and (bio)chemical properties of oil(s) allowing for better embryo microenvironment control/encapsulation, supplement access and removal of toxic metabolites;

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

Further disclosure, objects, advantages and aspects of preferred and other embodiments of the present invention may be better understood by those skilled in the relevant art by reference to the following description of embodiments, which are given by way of illustration only, and thus are not limitative of the disclosure herein.

### BRIEF DESCRIPTION OF THE DRAWINGS

Further disclosure, objects, advantages and aspects of preferred and other embodiments of the present invention may be better understood by those skilled in the relevant art by reference to the following description of embodiments taken in conjunction with the accompanying drawings, which are given by way of illustration only, and thus are not limitative of the disclosure herein, and in which:

FIG. 1 illustrates an analysis of pluripotency markers Genea018, overlaid with Sage™ IVF Oil (fused image) in accordance with a preferred embodiment of the invention.

FIG. 2 illustrates an analysis of pluripotency markers in Genea018, overlaid with Compound 1 (fused image) in accordance with a preferred embodiment of the invention.

FIG. 3 illustrates as analysis of pluripotency markers in Genea018, overlaid with Compound 2 (fused image) in accordance with a preferred embodiment of the invention.

FIG. 4 illustrates an analysis of pluripotency markers in Genea018, overlaid with Compound 3 (fused image) in accordance with a preferred embodiment of the invention.

### DETAILED DESCRIPTION

In contrast to the use of mineral oils in the prior art, with preferred embodiments, the inventor proposes to use well-characterized synthetic polymer(s), synthetic or natural monomeric small molecule organic compound(s) or appropriate mixtures thereof with additional components including but not limited to other small molecules, polymers, antioxidants, nutrients, biomolecules including but not limited to nucleotide and nucleotide sequences, oligomers (ex., DNA, RNAs, their fragments and/or synthetic analogues), amino acids, peptides, proteins, antibodies and other favorable biomolecules displaying well-defined and controlled chemical composition, embryo-compatible (bio)physical and (bio)chemical properties, stability and easily available commercially as food-grade or medical device-grade (H-1 or higher, as per National Sanitation Foundation categorization) inert ‘silent’ media component for embryo or general in vitro/ex vivo protein and cell biology. Furthermore, physical, chemical and biological properties of these and related compounds could be further optimized synthetically or via additives in order to attain the desired physiological and clinical outcome appropriate for biological samples.

In a first stage of a representative calibration approach, the inventor has identified several polyalpha- or related polymers and relevant (co)polymers from commercial sources. Representative examples comprise the following:

Food Grade Synthetic Oil ISO 220, 55 Gal (http://www.grainger.com/product/CRC-Food-Grade-Synthetic-Oil-ISO-12G564)
Food Grade Silicon Spray (Weston Brand™, http://www.schaefferoil.com/276-food-grade-lube.html)
Summit Syngear™ Food Grade (FG) fully synthetic lubricants (http://www.klsummit.com/products/lubricant/syngear-fg-series)
Sprayon™ LU209 Food Grade Synthetic Oil (http://www.sprayon.com/product-categories/industrial-lubricants/food-grade-synthetic-oil-aerosol-lu209)
Lubriplate™ NSF H1 Registered Food Machinery Lubricants (https://www.lubriplate.com/Products/NSF-H-1-Registered-Food-Machinery-Lubricants.aspx)

For embodiments, the key selection criteria include:
1. A true organic small-molecule oil with tentative molecular weight MW<5,000D. The preferred candidate is a well characterized ‘inert’ monomer or polymer as exemplified, including but not limited to, long chain alkanes, cycloalkanes, long chain aliphatic alcohols, ethers, esters, amides, lactones, lactams, etc.
2. A specific set of biophysical, chemical, stability, toxicity criteria including density, viscosity (kinetic and dynamic), surface tension, etc
3. Synthetic or well defined naturally originating oils, which are generally recognized as safe, ie GRAS;
4. Well-defined chemical composition and (micro)impurities including both organic and inorganic substances;
5. Chemical stability and inertness, such as to sunlight, air/oxygen and, temperature. Biological stability/inertness, embryo and/or relevant oligonucleotide, protein, cell, tissue, intact organism-isolation/encapsulation potential;
6. Physical properties compatible with objects defined in the selection criterion 5. These comprise volatility, melting point, boiling point, standalone safety, flashpoint, molecular weight, viscosity range, surface tension, gas/liquid diffusion/miscibility potential, etc.;
7. Physical properties which allow the compound to prevent evaporation, therefore allowing it to be used as an overlay for culture media to prevent osmolality, temperature and pH deviations;
8. Feasibility of access, modification, synergistic potential with favorable additives and ease to use/operate;
9. Commercial feasibility.

The identified lead candidates for an encapsulating overlay that satisfy the abovementioned criteria may be further evaluated in stem cell and embryonic development assays as per standard protocol described for paraffin/mineral oil(s) to further select candidates. In addition, adding chemically/biologically inert additives to the encapsulating overlay comprising small molecule-based monomer compounds and/or related fully synthetic compound(s) in order to further optimize their physical/biological properties is envisaged. These additives include but are not limited to respective surfactants, reactive oxygen species/metabolite scavengers and/or nutrients, gene-altering antisense DNA or RNA sequences, peptides, proteins, peptides, peptoids and other favorable molecules.

Lead candidate compounds which include additives could also be used for other screening and biological manipulations involving element-sensitive proteins, cells, cell cultures, multi-origin tissues, tissue cultures and intact organisms.

Numerous single small molecule-based compounds are readily available commercially and may be further customized to match specific embryo culture specifications via a variety of synthetic procedures. Chemical classes which could be utilised include hydrocarbons of various lengths, both branched, linear and cyclic, as well as modified hydrocarbons (including, but not limited to, fluorocarbons).

In one embodiment, Polyalphaolefins (PAOs) may be utilised. PAOs are readily available commercially and may be further customized to match specific embryo culture specifications via a variety of synthetic procedures. In this respect, the following listed references may be utilised for such procedures:
1. Rudnick, L. R. “Polyalphaolefins,” Chemical Industries (Boca Raton, Fla., United States) (2013), 135 (Synthetics, Mineral Oils, and Bio-Based Lubricants), 3-40.
2. Gee, J. C. et al. “Behavior of protonated cyclopropyl intermediates during polyalphaolefin synthesis: Mechanism and predicted product distribution,” Journal of Physical Organic Chemistry (2012), 25(12), 1409-1417.
3. Yu, X. et. al “Synthesis of polyalphaolefins on AlCl3/TiCl4 catalyst,” China Petroleum Processing and Petrochemical Technology (2012), 14(2), 55-59.
4. Azizov, A. H., et al “Advancement in the synthesis &production of polyalphaolefin synthetic oils: I. synthesis of poly-α-olefin synthetic oils by catalytic oligomerization of α-olefins with acidic & complex catalysts,” Neft Kimyasi va Neft E'mali Proseslari (2010), 11(1), 53-78.
5. Azizov, A. H., et al “Advancement in the synthesis and production of polyalphaolefin synthetic oils: II. Synthesis of polyalphaolefin synthetic oils by catalytic oligomerization of alpha-olefins in the presence of ionic liquid catalysts,” eft Kimyasi va Neft E'mali Proseslari (2010), 11(2), 163-182.
6. Tsvetkov, O. N. “Catalytic processes in the manufacture of poly α-olefins,” Kataliz v Promyshlennosti (2002), (6), 33-40.
7. Shubkin, R. L. “Polyalphaolefins,” Chemical Industries (Dekker) (1993), 48 (Synthetic Lubricants and High-Performance Functional Fluids), 1-40. Galli, R. D. “A New Synthetic Food Grade White Oil,” Lubrication Engineering (1982), 38(6), 365-72.

It is worth noting that multiple publications describe utility of polyalphaolefins (PAOs) as food-grade (H-1, as designated by the National Sanitation Foundation) in the last 2 decades. Hence, there is now a clear indication that PAO's are a safe material in the food industry and, by the inventor's inference and investigation, PAO's may be safely and validly synthesised as candidates for the synthetic compound utilised in embodiments of the present invention.

It is further anticipated that the disclosed embodiments of the invention could be used in a broader array of in-vitro, cell-based, tissue culture and in-vivo assays involving intact organisms. Specifically, the aforementioned inert compounds may be applied directly to insulate the actual screening media (including, but not limited to (micro)drop(s) in the screening well of 96-, 384-, 1536-well or any alternative plate, open or closed channel microfluidics devices, etc) from exposure to the environment and/or to maintain key screening parameters including volume, composition, osmolarity, nutrient content, etc. The invention is of particular benefit to screening biological objects, cells, tissues, and organisms that may be sensitive to elements using any conventional, medium- or high throughput dispensing technique. Additional benefit(s) provided by the disclosed ‘inert compounds’ used as overlaying encapsulants for biological samples may also include complete transparency to the common non-intrusive light-absorbance, emission, scattering detection techniques including UV-vis, near-IR, far-IR spectroscopy, electron paramagnetic resonance and biophysical platforms including but not limited to surface plasmon resonance (SPR), thermal melt and other assay techniques. Representative examples include, but are not limited to:
i) in-vitro manipulation (storage, dispensing, screening) of air/oxygen, UV light-sensitive, osmolarity, pH proteins. By way of example, biomolecules comprising multiple SH and/or S—S bonds as exemplified by the family of cytokine and chemokine proteins; proteins/enzymes featuring coordinated metal(s) including but not limited to Zn, Mg, Mn, Cu, Fe as exemplified by the epigenetics targets including but not limited to histone deacetylases, histone demethylases, histone acetylases, metalloproteinases, hydrolases, etc;
ii) in-vitro manipulation of any nucleotide sequences including but not limited to endogenous, intact, fragmented, chemically modified DNA, mRNA, shRNA, siRNA, miRNAs as exemplified by q-PCR, transfection and gene editing techniques;
iii) cell-based screening including but not limited to any manipulations of stem cell(s) or relevant derivative(s) thereof as exemplified by human/animal-derived embryonic stem cells, induced pluripotent stem cells, immediate or advanced (differentiated) derivatives of these, genetically manipulated derivatives of stem cells, etc;
iv) any cell culture in a relevant treatment receptacle including but not limited to microtiter, midi- or macro-plates, microfluidics devices, stationary, suspended drop, flow systems or similar. These cell cultures include but are not limited to human/animal embryos/cells, specific differentiated human/animal cells as exemplified by an organ/tissue derived neurons, cardiomyocytes, fibroblasts, hepatocytes, renal cells; stem cells/primary cells/cancer cells/otherwise immortalized cells, genetically altered/engineered cells, stably and/or transiently transfected cells, cells labelled with fluorescent, radio, radical and/or other detection functionalities, etc.
v) functional/phenotypic screening using relevant healthy, diseased, modified or transfected cell lines as exemplified by differentiation, proliferation, migration, adhesion, motility, chemotaxis and other cellular assays;
vi) screening using intact or suspended tissue (for example, matrigel-based clonogenic assay(s)) of interest and/or intact organisms as exemplified by the sea urchin embryo, zebrafish and other in-vivo assay(s) where maintenance of homeostasis is of critical importance.

### Preliminary Experimental Data on Use of Synthetic Compounds as Overlay for Cell and Embryo Culture

Experimental results from trials conducted by the inventor involving embodiments of the invention are as follows:

### Aims

To perform preliminary tests about the feasibility of three synthetic compounds in cell and embryo culture.

### 1. Experiment 1—Stem Cells

1.1. Experimental Procedure

Materials:

Test compounds:

Compound 1

Compound 2

Compound 3

According to brochures available from the manufacturer, the selected test compounds are hydraulic and lubricating compounds based on high-purity hydrocarbons with paraffinic synthetic oil. They are a combination of basic oils and additives, which can be used in the food processing industry. In particular, Compound 1 is a mixture of short, long and branched, fully saturated hydrocarbons with no presence of aromatic groups. An example of a suitable candidate that would fall within the scope of Compound 1 is found in the source: ‘TURMOSYNTH™ VG series Technical Information’ and technical information is presented in the following Table 1.

TABLE 1

| | TURMOSYNTH VG | | | | | | | | | | |
|---|---|---|---|---|---|---|---|---|---|---|---|
| Technical Data | 15 | 32 | 46 | 68 | 100 | 150 | 220 | 320 | 460 | 680 | 1500 |
| NSF/H1- | 127133 | 132163 | 139108 | 127132 | 127138 | 127139 | 132161 | 132161 | 127122 | 132162 | 127131 |
| registration | | | | | | | | | | | |
| Colour | Clear, nearly colourless | | | | | | | | | | |
| Density | 0.85 | 0.86 | 0.87 | 0.88 | 0.88 | 0.88 | 0.88 | 0.88 | 0.88 | 0.89 | 0.89 |
| at +20° C. | | | | | | | | | | | |
| (g/cm³) | | | | | | | | | | | |
| Temperature | −10° C. to +100° C., higher viscosities a short time up to +120° C. | | | | | | | | | | |
| range | | | | | | | | | | | |
| Viscosity(mm²/s) | | | | | | | | | | | |
| DIN EN ISO | | | | | | | | | | | |
| 3104 | | | | | | | | | | | |
| at +40° C. | 16.3 | 33.6 | 43.5 | 67.6 | 100.8 | 142.7 | 227.2 | 323 | 459.9 | 692 | 1504 |
| at +100° C. | 3.6 | 5.7 | 6.7 | 9.0 | 11.7 | 13.8 | 23.0 | 32 | 42.6 | 59.1 | 117.7 |
| Viscosity index | 102 | 109 | 107 | 108 | 104 | 116 | 125 | 138 | 144 | 149 | 174 |
| DIN ISO 2909 | | | | | | | | | | | |

Applications
Machines in the food industry with oil lubrication, like hydraulics, gears, bearings, chains, spindles, levers and links.

The manufacturing process for the synthesis of the three selected compounds comprises the combination of specific raw materials within a mixing vessel. This differs from the mineral oil process which involves the fractional distillation of a natural product (crude oil) and purification to reach the finished product.

Human embryonic stem cell lines (hESC) lines
Manually passaged, cultured on mouse fibroblast feeder layers
Cells cultured on Nunc IVF 1-well dishes, in KnockOut™ Serum Replacement¹⁶ (‘KSR’)-media in large incubator at +37° C. at 6% CO₂, 5% O₂ and 89% N₂ ¹⁶ See https://www.thermofisher.com/order/catalog/product/10828028

The used hESC lines were manually passaged human embryonic stem cell lines. The dishes for the experiment were dishes remaining after manual cutting and removal of hESC colonies 8 days after previous passaging. The remaining colonies are still able to be cultured, although eventually they start to differentiate and lose pluripotency, and even degenerate if not adequately fed. Each dish contained cells from a different cell line and passage number.

Control cultures were plated and overlaid with Sage™ IVF Oil, which is regularly used in embryo culture. This was to allow comparisons to be drawn between the ability of cells to be cultured under Sage™ IVF Oil and test compounds.

The KSR media had been changed for passaging, so the experiment started with 1 ml of fresh media in each dish. For the actual experiment all wells were layered with 1 ml of test oil that had been equilibrated overnight in a 20% O₂ and 5% CO₂ incubator at 20% O₂. The dishes were then cultured further in a low oxygen incubator (6% CO₂, 5% O₂ and 89% N₂) for overnight.

The media on dishes were replaced with fresh KSR media the following day and then left without media change over the next two days. Cell appearance was observed at dish preparation, after overnight culture (at 1 day), after 3 more days of culture (at 4 days) and at day 7, when immunohistochemical staining was also performed using three antibody markers (SSEA-4, Oct-4 and Nanog). These particular molecular markers were used because their presence verifies the pluripotent status of the stem cells. A down-regulation of either SSEA-4, Oct-4 or Nanog would signify that cells are differentiating and are no longer pluripotent, meaning that the hESCs are under stress. All dishes were discarded at that point and the experiment concluded.

1.2. Results & Discussion

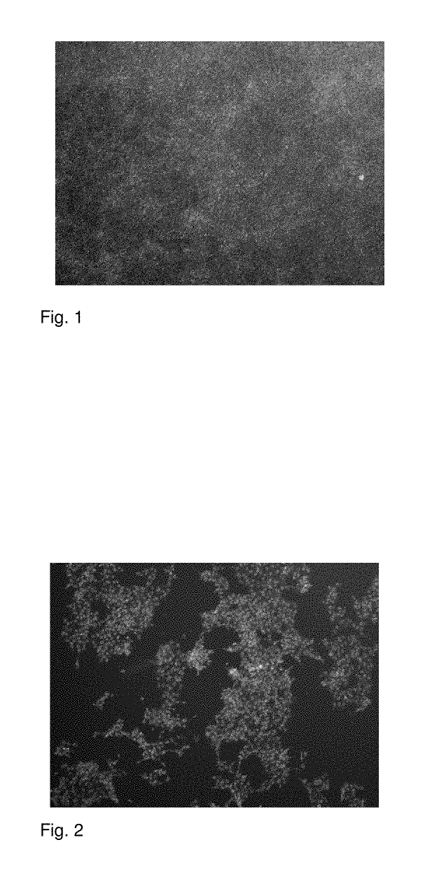

hESCs continued to grow under all test compounds and displayed pluripotency on day 7. FIGS. 1 to 4 show the cells cultured under an overlay of the Sage™ IVF oil, Compound 1, Compound 2 and Compound 3, respectively, after 7 days of culture. The development of cells cultured underneath an overlay of Compound 3 is very similar to those cultured under an overlay of Sage™ IVF Oil (the control). Cells cultured underneath an overlay of Compound 1 and Compound 2, although not forming a perfect monolayer, did not simply degenerate and therefore, Compound 1 and Compound 2 were not immediately cytotoxic. However, they may not provide an environment for cell proliferation which is as suitable as Compound 3 or Sage™ IVF oil.

No detailed information about proliferation rate or cellular differentiation rates were obtained in this experiment, as the intention was only to test preliminary reaction of cells to test compounds.

The cells used in this experiment were hESC lines that were maintained and passaged as colonies rather than single cells. This method of culturing is still the method used at initial derivation of new lines from human embryos, and is also used for early passages to best maintain the integrity of stem cell lines and to avoid chromosomal deviations that may arise in later passages, especially if passaged enzymatically as single cells.

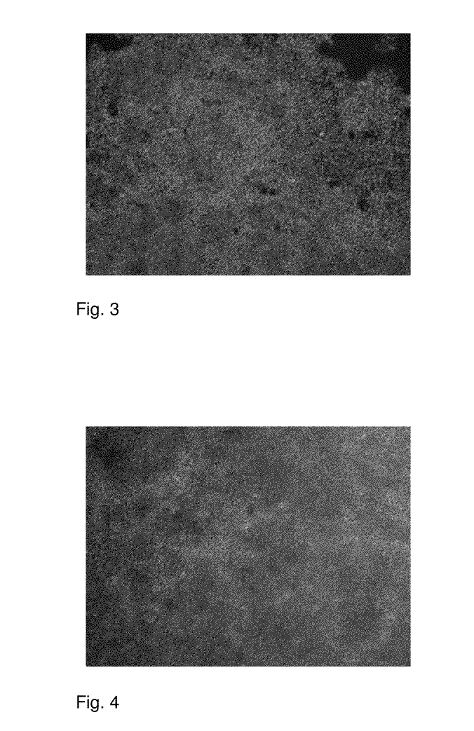

FIG. 1 illustrates an analysis of pluripotency markers Genea018, overlaid with Sage™ IVF Oil (fused image). FIG. 2 illustrates an analysis of pluripotency markers in Genea018, overlaid with Compound 1 (fused image). FIG. 3 illustrates as analysis of pluripotency markers in Genea018, overlaid with Compound 2 (fused image). FIG. 4 illustrates an analysis of pluripotency markers in Genea018, overlaid with Compound 3 (fused image).

1.3. Conclusions

The stem cells survived and grew when applied with an overlay of all compounds. Cells cultured under an overlay of Compound 3 displayed proliferation similar to control (Sage™ IVF Oil). Cells cultured under an overlay of Compound 1 and Compound 2, although not forming a complete monolayer as could be seen for cells under Compound 3 and Sage™ IVF Oil, still did experience growth and were not dead after 7 days of culture. Therefore, it is clear that none of the compounds were cytotoxic, and that all allowed cell proliferation. In addition, it is possible to see from the images showing the fluorescence of all three pluripotency markers in each of the samples that the hESCs maintained their pluripotency after 7 days of culture under all test and control compounds.

### 2. Experiment—Embryos

2.1. Experimental Procedure

Materials:

Test compounds:

Compound 1

Compound 2

Compound 3

Sage™ IVF Oil (CONTROL)

Single-Step Human Embryo Culture Medium

Falcon® 60 mm dishes

Mouse embryos at 2PN stage.

60 mm Falcon® petri dishes were prepared with Single-Step Human Embryo Culture Medium and Sage™ IVF Oil (control compound), Compound 1, Compound 2 or Compound 3 (test compounds) as per routine culture of mouse embryos. Briefly, 9×20 μl drops were prepared under 6 ml of control or test compound, and left to equilibrate in a Cook MINC™ incubator¹⁷ at +37° C. at 6% CO₂, 5% O₂ and 89% N₂ overnight. The next day (Day 1), embryos which had been classified as being 2PN stage were placed into the drops following removal of cumulus cells. No more than ten embryos were placed in each drop. Embryos were then assessed for development as per routine mouse embryo assay (MEA) protocol on days 2, 5, 6 and 7. ¹⁷ See https://www.cookmedical.com/products/wh_minc_1000_webds/

2.2. Results & Discussion

Embryo development and quality was comparable between the control and Compound 3 at all stages of assessment. Embryos degenerated prior to their first cell division when media was overlaid with Compound 1 or Compound 2. Therefore, although Compound 1 and Compound 2 were not toxic to stem cells, they are both clearly toxic to embryos.

2.3. Conclusions

Overlaying the culture media with Compound 3 enabled embryos to fully develop to blastocyst stage. The amount of embryos which developed and their quality was not statistically different between control and test groups. Overlaying the culture media with Compound 1 or Compound 2 caused almost instant embryo degeneration.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, means-plus-function clauses are intended to cover structures as performing the defined function and not only structural equivalents, but also equivalent structures. For example, although a nail and a screw may not be structural equivalents in that a nail employs a cylindrical surface to secure wooden parts together, whereas a screw employs a helical surface to secure wooden parts together, in the environment of fastening wooden parts, a nail and a screw are equivalent structures.

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

1. An overlay encapsulant for an in vitro cell culture comprising a synthetic compound.

2. An overlay encapsulant according to claim 1 wherein the cell culture comprises one or more cells in a culture media.

3. An overlay encapsulant according to claim 2 wherein the one or more cells comprises at least one or a combination of:
ovum;
zygote;
embryo;
animal/human-derived embryonic stem cell(s);
relevant pluripotent derivative(s) and/or differentiated progeny;
intact or dispersed tissue and/or intact organism.

4. An overlay encapsulant according to claim 1 wherein the synthetic compound is a synthetic small molecule composition exhibiting unequivocal chemical composition as identified via conventional analytical techniques within limits of detection and comprising one or a combination of.
synthetic monomer(s);
oligomers or polymers;
chemical derivatives and/or copolymers of polyalphaolefins,
each exhibiting specific chemical, biophysical and spectroscopic properties.

5. An overlay encapsulant according to claim 1 wherein the synthetic compound comprises at least one hydrocarbon.

6. An overlay encapsulant according to claim 1 wherein the synthetic compound comprises a modified hydrocarbon.

7. An overlay encapsulant according to claim 6 wherein the modified hydrocarbon comprises a fluorinated hydrocarbon.

8. An overlay encapsulant according to claim 1 wherein the synthetic compound comprises one or a combination of long-chained, short-chained and cyclic hydrocarbons.

9. An overlay encapsulant according to claim 8 wherein the synthetic compound comprises a combination of long-chained, short-chained and cyclic hydrocarbons in the mixture of 45% long-chained, 38% short-chained and 17% cyclic, respectively.

10. A method for temporary encapsulation of an in vitro cell culture comprising the step of overlaying the cell culture with a synthetic compound.

11. A method for temporary encapsulation of at least one of protein(s), DNA, RNA sequence(s), relevant construct(s) and/or derivative(s), chemically-modified or derived analogues thereof for in vitro, ex vivo and/or in vivo manipulation thereof, the method comprising the step of:
overlaying a manipulation and/or screening media utilised in the in vitro, ex vivo and/or in vivo manipulation with a synthetic compound.

12. An overlay encapsulant according to claim 1 wherein the synthetic compound comprises a well-defined chemical compound as described by conventional analytical techniques comprising one of NMR, HPLC, LCMS within the limit of detection wherein the compound is exemplified by one of:
i) regimented polymer with well-defined chemical and/or biophysical properties,
ii) small molecule,
iii) inert gas heavier than air.

13. An overlay encapsulant according to claim 1 which is adapted to monitor deviations in the properties and composition of media encapsulated thereby.

14. An overlay encapsulant according to claim 13 wherein the monitored properties and composition of encapsulated media comprise one or a combination of:
pH,
ammonia concentration,
osmolarity,
presence of reactive oxygen species, and
presence or volatile organic compounds.

15. An overlay encapsulant according to claim 1 in which the overlay encapsulant is adapted to be used as a supplement source comprising one or a combination of:
vitamins,
hormones,
growth factors,
nutrients,
protectants,
RedOx traps,
amino acids and their derivatives,
peptoids,
peptides,
proteins,
antibodies and relevant derivatives, fragments and full length oligonucleotides and their synthetic derivatives.

16. An overlay encapsulant according to claim 1 in which the overlay encapsulant is adapted to be used for screening or biological manipulations involving one or more of:
element-sensitive proteins,
cells or cell cultures,
multi-origin tissues or tissue cultures, and
intact organisms.

17-18. (canceled)
